(19) United States
(12) Patent Application Publication
(10) Pub. No.: US 2009/0232888 A1
(43) Pub. Date: Sep. 17, 2009

(54) SUSTAINED DELIVERY OF ANTIBIOTICS

(75) Inventors: Chin-Chih Chiang, West Covina, CA (US); Remy Chen, Taichung City (TW)

Correspondence Address: HOGAN & HARTSON LLP;IP GROUP, COLUMBIA SQUARE, 555 THIRTEENTH STREET, N.W., WASHINGTON, DC, 20004, US

(73) Assignee: ORIENT PHARMA, Dounan Town (TW)

(21) Appl. No.: 12/403,896

(22) Filed: Mar. 13, 2009

Related U.S. Application Data
(60) Provisional application No. 61/064,631, filed on Mar. 17, 2008.

(51) Int. Cl. A61K 9/52; A61K 31/7028; A61P 31/00

(52) U.S. Cl. 424/457; 514/35

(57) ABSTRACT

The present invention is directed to compositions for the sustained delivery of an antibiotic, for example vancomycin, to achieve desirable release profiles. This application is also directed to methods of using the compositions and processes for manufacturing the compositions.

### BACKGROUND

The administration of antibiotics to patients often requires frequent dosing, refrigeration of medicines, and other factors that can effect patient compliance with dosing regimens and/or the effectiveness of the medication in treating a disease.

For example, vancomycin can require frequent dosing to be effective. Vancomycin is a tricyclic glycopeptide antibiotic derived from Amycolatopsis orientalis (formerly Nocardia orientalis) which is indicated for the treatment of serious, life-threatening infections by Gram positive bacteria which are unresponsive to other antibiotics. Traditionally, vancomycin has been marketed in two dosage forms, injectable formulations and oral capsules.

Vancomycin often needs to be administered intravenously (IV) for systemic therapy since it does not cross the intestinal lining. It is a large hydrophilic molecule which partitions poorly across the gastrointestinal membrane. The injectable vancomycin product is a frozen, iso-osmotic, sterile, nonpyrogenic premixed 100 mL or 200 mL solution containing 500 mg or I gam vancomycin in its hydrochloride form. Each 100 mL of solution contains approximately 5 gams of dextrose hydrous USP. The pH of the solution may be adjusted with hydrochloric acid and/or sodium hydroxide. Thawed solutions can have a pH in the range of 3.0 to 5.0. After thawing the solution to room temperature, it is intended for intravenous use only. It has been reported that vancomycin solutions were stable for at least 58 days at 4° C. based on a shelf-life of 90% residual potency. See, e.g., “Long-term stability of vancomycin hydrochloride in intravenous infusions” published in the Journal of Clinical Pharmacy and Therapeutics, Volume 22 Issue 5 Page 353-356, November 1997.

Vancomycin can also be orally administered to treat specific conditions, e.g., pseudomembranous colitis, where the antibiotic does not need to cross the intestinal lining but instead remains in the gastrointestinal tract. One orally administered form of vancomycin is the Vancocin® capsule. Each capsule contains either 125 mg or 250 mg of vancomycin base and is approved by the FDA for the treatment of entero-colitis. Oral administration of a Vancocin capsule shows poor systemic absorption of vancomycin because the oral bioavailability of vancomycin is negligible. The inactive ingredient in Vancocin® capsules is polyethylene glycol. Release of vancomycin from these capsules shows a fast release profile (See FIG. 1). According to the Physicians' Desk Reference (PDR), Vancocin® capsules are to be administered 3 to 4 times a day. This frequent daily administration can be inconvenient for patients and may reduce patient compliance.

### SUMMARY

The application is directed to a pharmaceutical composition containing at least one antibiotic, for example vancomycin, in a sustained release dosage form that contains at least one immediate release component and at least one sustained release component. These dosage forms allow for a reduction in the frequency of the antibiotic that needs to be administered to have its desired therapeutic effect.

Embodiments of the invention described herein are also directed to methods for preparing a capsule formulation of an antibiotic by filling a capsule with at least one immediate release component and at least one sustained release component.

Further embodiments of the invention described herein are also directed to methods of filling a capsule with a barrier in between one immediate release component and one sustained release component.

Further embodiments of the invention described herein are directed to methods of treating diseases, for example entero-colitis, by administering an effective amount of any pharmaceutical composition described herein to a subject in need thereof.

### BRIEF DESCRIPTION OF THE FIGURES

FIG. 1 illustrates a dissolution profile of vancomycin HCI from Vancocin® capsules.

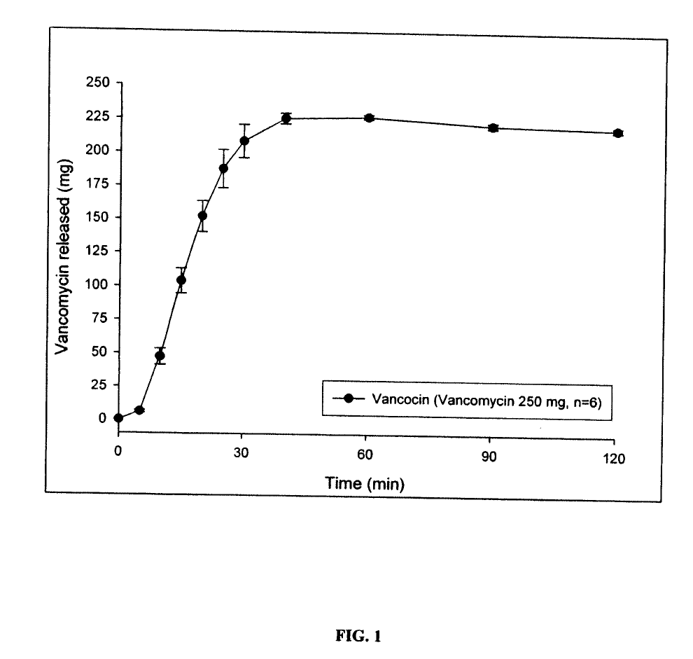

FIG. 2 illustrates the dissolution profiles of vancomycin HCI from two example embodiments of the present invention: formulation 1 and formulation 2.

FIG. 3 illustrates a comparison of the dissolution profiles of vancomycin HCI from Vancocin® to another example embodiment of the present invention, formulation 3.

### DETAILED DESCRIPTION

Embodiments of the present invention are directed to compositions for the controlled release of antibiotics and methods of using those compositions. These compositions have desirable antibiotic release profiles that can allow patients to take them less per day than other formulations. The application further provides methods of preparing the compositions and processes for manufacturing the compositions.

The desirable sustained release profiles can be obtained using a single dosage form, e.g., a capsule. These desirable release profiles can be achieved by including immediate release and sustained release components within a single dosage form, e.g., a capsule.

The dosage forms described herein can be used to deliver antibiotics locally and/or systemically. The term “antibiotic” encompasses any pharmaceutically acceptable compound that can inhibit the growth of or destroy bacteria and/or other microbes, regardless of whether the compound is produced in a microorganism or produced synthetically. This term encompasses disinfectants, antiseptics, and any other antimicrobial compounds. For example, the term “antibiotic” encompasses penicillin and all its derivatives.

In some embodiments, the antibiotic is delivered locally within the gastrointestinal tract. For example, the antibiotic can be vancomycin, which is retained in the gastrointestinal tract after oral administration. The term “vancomycin” refers to the tricyclic glycopeptide antibiotic derived from Amycolalopsis orientalis (formerly Nocardia orientalis). In some embodiments, the hydrochloride form of vancomycin is used. This form of vancomycin has a molecular formula of C₆₆H₇₅Cl₂N₉O₂₄. HCl and a molecular weight of about 1485.73. One of skill in the art will appreciate that other forms of vancomycin can be used in the embodiments described herein.

Suitable antibiotics for local delivery to the gastro intestinal tract include, but are not limited to, vancomycin, teicoplanin, ramoplanin, difimicin, kanamycin, neomycin (or other aminoglycosides) and colistin.

In some embodiments, the antibiotic administered can act systemically. For example, suitable antibiotics for systemic delivery include, but are not limited to, beta-lactam antibiotics, azithromycin, clarithromycin, erythromycin (or other macrolide antibiotics), ciprofloxacin, flucloxacillin, ofloxacin, norfloxacin, enoxacin (or other fluoroquinolones), rifaximin, and metronidazole. In some embodiments, a locally acting antibiotic can be administered with a systemic antibiotic.

The dosage forms can comprise an antibiotic in an amount from about 50 mg to about 800 mg, about 150 mg to 500 mg, or about 175 mg to about 400 mg. In some embodiments, these amounts of antibiotic can be within a single pharmaceutically acceptable capsule. In some embodiments, the antibiotic is vancomycin HCI.

The compositions, i.e. the dosage forms, described herein can include an immediate release component and a sustained release component. Either of these components can be prepared using hot melt methods and are each specifically designed to predictably and consistently deliver a desired amount of an antibiotic over a desired time period. For example, the dosage form can deliver a minimum inhibitory concentration of vancomycin for up to about 4, about 6, about 12, about 18, or about 24 hours.

The term “immediate release component” refers to the portion of the dosage form that releases substantially all of its active ingredient (e.g., an antibiotic) within 5 minutes to about 60 minutes. The term “substantially all,” when used in reference to the amount of antibiotic released, means over 70%, 85%, 90%, 95%, or 99% of the amount of antibiotic in the formulation component has been released.

The term “sustained release component” refers to the portion of the dosage form that releases substantially all of its active ingredient (e.g., an antibiotic) within about 180 minutes to about 600 minutes. The antibiotic release times for the immediate and/or sustained release components can be measured using standard dissolution assays known to one of skill in the art and as described herein. Examples of release times observed for vancomycin formulations are illustrated in the figures and described in the examples to follow.

The compositions described herein can have at least one immediate release component and at least one sustained release component in a single dosage form. Some embodiments of this invention are capsules. The capsule used can be a hard gelatin, soft gelatin, or other suitable capsule.

The immediate release component can contain hot-melt excipients that are dissolved in a media of pH<5. The hot-melt excipients can include, but are not limited to, polyethylene glycols (PEGs) and/or polyethylene glycol esters.

Suitable polyethylene glycol esters are commercially available as Gelucire®. Gelucire® is a semi-solid mixture of glycerol and PEG1500 esters of long fatty acids. There are at least two grades of Gelucire®, i.e., Gelucire® 40/14 and Gelucire® 50/13. The suffixes of 44 and 14 or 50 and 13 refer to, respectively, its melting points and its hydrophilic/lipophilic balance (HLB).

The immediate release component comprises an antibiotic, for example vancomycin, and one or more hot-melt excipients. The amount of vancomycin in the immediate release component can be from about 15% to about 85% (w/w), about 30% to about 60% (w/w), or about 35% (w/w) to about 50% (w/w). For example, one embodiment of an immediate release component can include 37.2% vancomycin HCI (w/w) and 62.8% (w/w) PEG3500.

The compositions described herein also have a delayed release component. The sustained release formulation can contain hot-melt excipients and/or pH sensitive dissolving materials that are dissolved in a media of pH>5. Suitable materials include, but are not limited to, sodium alginate, polyethylene glycols, and polyethylene glycol esters.

The delayed release formulation component can contain an antibiotic, such as vancomycin, hot-melt ingredients, and excipients. The percentage of antibiotic in the sustained release formulation can vary from about 15% to about 85% (w/w), about 30% to about 60% (w/w), or about 35% (w/w) to about 50% (w/w). For example, an embodiment of the sustained release component can contain 37.2% (w/w) of vancomycin HCI, 48.4% (w/w) of Gelucire®, and 14.4% (w/w) of sodium alginate.

The compositions described herein can also have a barrier component in between one immediate release component and one sustained release component in a single dosage form. Some embodiments of this invention are capsules. The capsule used can be a hard gelatin, soft gelatin, or other suitable capsule.

The barrier component can contain hot-melt excipient that shows low solubility to the active ingredients and/or excipients which are used in the immediate release component and sustained release component.

Suitable barrier component can include, but are not limited to paraffin and/or wax.

The dosage forms described herein are designed to provide desirable antibiotic release profiles. These release profiles can be achieved, for example, by using a single dosage form having an immediate release component that is quickly dissolved in the acidic gastric environment in combination with a sustained-release component containing antibiotic that is slowly released over a longer time period. In some embodiments, this combination can act to prolong the release of vancomycin in patients allowing fewer dosings to be administered to the patient to achieve the desired concentrations in gastro-intestinal tract.

As one of skill in the art will appreciate, the desired release profile and concentrations of an antibiotic can be measured using the assays described herein and also using the following parameters: (1) the ratio of the peak concentration (Cmax) to the minimum inhibitory concentration (MIC); (2) the ratio of the area under the concentration-time curve (AUC) to MIC; and (3) the time the concentration exceeds the MIC. These factors are important in evaluating the clinical efficacy of antibiotics.

For example, vancomycin activity is considered to be time-dependent; antimicrobial activity depends on the duration that the drug level exceeds the MIC of the target organism. Keeping the drug level exceeding MIC using a modified release of vancomycin to continuously deliver the drug into the target sites can result in a reduced frequency of dosing without disrupting the clinical benefits while improvising patient compliance with the treatment regimen.

For example, in some embodiments the dosage forms of the present invention release about 5% to about 50% of the total vancomycin in the dosage form within sixty minutes. The remaining 50% of the total vancomycin can be released in about 120 minutes to about 480 minutes.

Example dosage forms are designed to release about 20% to about 80%, about 40% to about 75%, or about 50% to about 70% of an antibiotic, such as vancomycin, in the first two hours and the remaining antibiotic in about 12 hours.

The selection of the hot-melt excipient used in the dosage forms described herein can be used to provide the desired release profile. These pharmaceutically acceptable excipients function not only as carriers for the vancomycin, but also can be selected to modify the release properties of vancomycin in the gastrointestinal tract.

One non-limiting embodiment of the present invention is as follows:

A) The immediate release component includes vancomycin, the active ingredient, homogeneously dispersed into a water soluble material, such as polyethylene glycol, and an optional ingredient such as dextrose. The percentage of vancomycin HCI in the immediate release component of the dosage form can be from about 30-80% by weight, preferably about 40-50%. The percentage of polyethylene glycol in the immediate release component of the dosage form can be from about 30-80% by weight, preferably about 40-50%. The percentage of dextrose in the immediate release component of the dosage form can be about 10-40% by weight, preferably about 20-30%.

B) The sustained release component includes vancomycin, the active ingredient which can be homogeneously dispersed into a matrix. The matrix ‘can be composed of a hot-melting ingredient such as a hydrophilic compound, polyethylene glycol or a lipophilic compound, propylene glycol fatty esters, and an optional pH-sensitive polymer such as sodium alginate or sodium carboxymethyl cellulose. The percentage of vancomycin HCl in the sustained release component of the dosage form can be from about 30-80% by weight, preferably about 40-50%. The percentage of polyethylene glycol in the sustained release component of the dosage form can be about 20-40% by weight, preferably about 30-50%. The percentage of propylene glycol fatty ester in the sustained release component of the dosage form can be about 20-50%, preferably about 30-40%. The percentage of the pH-sensitive polymer such as sodium alginate in the sustained release component of the dosage form can be about 30-60%, preferably about 40-50%.

C) The barrier component includes paraffin or wax. It is an inert hot-melting ingredient. It can be filled while in a liquid form above its melting point on top of either immediate release component or sustained release component which has been prefilled into a capsule.

Embodiments of the present invention are also directed to methods of treating a disease or disorder in a subject using the compositions described herein. For example, the controlled release vancomycin formulations described herein can be used to treat entero-colitis.

As one of skill in the art will appreciate, the compositions of the present invention can be administered with to treat any disease for which an antibiotic or multiple antibiotics can be beneficial. For example, vancomycin, metronidazole, and/or bacitracin can be administered in a single dosage form, or in multiple dosage forms, to treat antibiotic-associated pseudo-membranous colitis (AAPMC).

For vancomycin and other antibiotics, the compositions can be administered once or twice daily, depending on the patient's needs. The daily dosage of vancomycin, or other antibiotic administered, can be readily determined by a physician using well known methods.

Embodiments of the present invention are also directed to methods of preparing a sustained release formulation of an antibiotic, for example, vancomycin. These methods can include filling a pharmaceutically acceptable capsule with at least one immediate release component and at least one sustained release component, with each component containing a specified amount of antibiotic.

Embodiments of the present invention are also directed to methods of preparing a sustained release formulation of an antibiotic, for example, vancomycin. The methods can include filling a pharmaceutically acceptable capsule with at least one immediate release component, one barrier component and at least one sustained release component, with each immediate release or sustained release component containing a specified amount of antibiotic while with no antibiotic in the barrier component.

These components may be added using two or more filling steps, depending on the dosage form desired. The immediate release component and the sustained release component can be prepared separately. In some embodiments, the sustained drug releasing layer is filled into the bottom of a capsule first and becomes a solid mass after cooling. The immediate release drug component is then filled on the top of the sustained drug release layer. However, as one of skill in the art will appreciate, the sustained release component and immediate release components can be added to the capsule in any order.

The sustained release formulations of vancomycin can be prepared by using the following non-limiting example of a multistage hot-melt process. Vancomycin HCl can be mixed with at least one pharmaceutical acceptable excipient that melts at elevated temperatures to form a homogeneous suspension. This suspension can be used in the immediate release component, the sustained release component, or mixtures of both, depending on the embodiment of the present invention. As one of skill in the art will appreciate, depending on the excipient selected, the temperature used, and the concentration of vancomycin HCl, the mixture liquid is not limited to a suspension and can also be, for example, a solution or an emulsion. Once prepared, this liquid is then filled into capsules at elevated temperatures.

In some embodiments, a two stage filling into capsules can be used to encapsulate the suspensions. The immediate release formulation element and the sustained release formulation element can be prepared and encapsulated separately in any order.

An example of a two stage filling process is as follows. One of the formulation elements is first filled into the capsules in a liquid form. The liquid formulation is then cooled down to room temperature to form a solid before the second formulation is filled in the same capsule. The formulation element having higher melting temperatures or higher viscosity is preferably to be filled first. In some embodiments, the immediate release component or the sustained release component is filled first.

After the first formulation has cooled within the capsule, a second fill can be made on top of the first. This second fill can include either a sustained release component, an immediate release component, or mixtures thereof.

The present invention also includes embodiments where more than two fill stages may be used. For example, the dosage form can have two, three, or four or more fill stages. These different stages can be used to layer the sustained release component and immediate release components in a variety of positions within the capsule.

In other embodiments, a three stage filling into capsules can be used. The immediate release formulation element and sustained release formulation element can be prepared and encapsulated separately in any order. The barrier element is filled in between both elements.

An example of a three stage filling process is as follows. One of the vancomycin containing formulation elements is first filled into the capsules in a liquid form. The barrier element is then filled on top of this first fill when it cools down. The other one of the vancomycin containing formulation is then filled on top of the barrier element.

While various embodiments of the present invention have been described above, it should be understood that they have been presented by way of example only, and not limitation. Thus, the breadth and scope of the present invention should not be limited by any of the above-described exemplary embodiments, but should instead be defined only in accordance with the following claims and their equivalents. All documents, including webpages, cited herein are incorporated in their entirety by reference.

### EXAMPLES

### Example 1

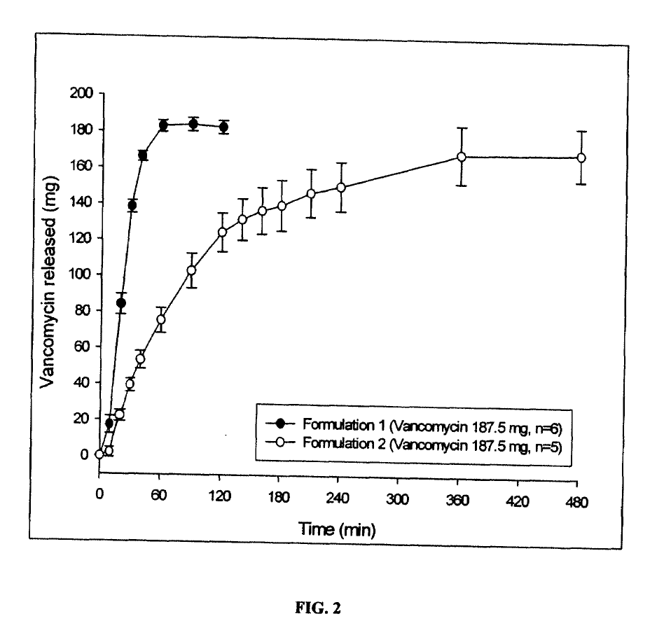

The following compositions have been prepared using the methods described herein. Dissolution results of example compositions are presented in FIG. 2.

TABLE 1
Formulation 1

| Dosage Form Component | Ingredients (in mg) | |
|---|---|---|
| Sustained Release Component | Vancomycin HC1 | 96 |
| | Gelucire | 139 |
| | Sodium Alginate | 42 |
| | TOTAL | 277 |
| Immediate Release Component | Vancomycin HC1 | 96 |
| | PEG3350 | 72 |
| | Gelucire | 35 |
| | TOTAL | 203 |

Example Manufacturing Process of Formulation 1

Step A (Preparation of the Sustained Release Element): 1. Melt 4.1744 grams of Gelucire® in a beaker in a hot plate stirrer at about 70° C. 2. Homogeneously disperse 2.8833 grams of vancomycin HCI in the same beaker while maintaining at the same temperature. 3. Add 1.2697 gm of sodium alginate in the same beaker and stir until homogeneous. 4. While warm, add 277 mg of the mixture into the bottom of a hard gelatin capsule. 5. Let the temperature of the fill drop to about room temperature.

Step B (Preparation of the Immediate Release Element): 6. Add and melt 2.1632 grams of PEG3350 and 1.0521 grams of Gelucire® in a beaker at a temperature of about 70° C. 7. Add and disperse 2.8901 grams of vancomycin HCI into the same beaker until homogeneous while maintaining the temperature at about 70° C. 8. Weigh 203 mg of the homogeneous suspension, and fill into the same hard gelatin capsule. 9. Allow the formulation to cool down to about room temperature. 10. Cap the capsules.

### Example 2

The following compositions have been prepared using the methods described herein. Dissolution results of example compositions are presented in FIG. 2.

TABLE 2
Formulation 2

| Dosage Form Component | Ingredients (in mg) | |
|---|---|---|
| Sustained Release Component | Vancomycin HC1 | 96 |
| | Gelucire | 124 |
| | Sodium Alginate | 40 |
| | TOTAL | 260 |
| Immediate Release Component | Vancomycin HC1 | 96 |
| | Gelucire | 29 |
| | PEG6000 | 1152 |
| | TOTAL | 240 |

Manufacturing Process of Formulation 2

Step A (Preparation of the Sustained Release Element): 1. Melt 6.2043 grams of Gelucire® in a beaker in a hot plate stirrer at about 70° C. 2. Homogeneously disperse 4.8005 grams of vancomycin HCl in the same beaker while maintaining at the same temperature. 3. Add 2.0147 gm of sodium alginate in the same beaker and stir until homogeneous. 4. While warm, add 260 mg of the mixture into the bottom of a hard gelatin capsule. 5. Let the temperature of the fill drop to about room temperature.

Step B (Preparation of the Immediate Release Element): 6. Add and melt 4.6032 grams of PEG6000 and 1.1790 grams of Gelucire® in a beaker at a temperature of about 70° C. Mix the solution until homogeneous. 7. Add and disperse 3.8647 grams of vancomycin HCl into the same beaker until homogeneous while maintaining the temperature at about 70° C. 8. Weigh 240 mg of the homogeneous suspension, and fill into the same hard gelatin capsule. 9. Allow the formulation to cool down to about room temperature. 10. Cap the capsules.

### Example 3

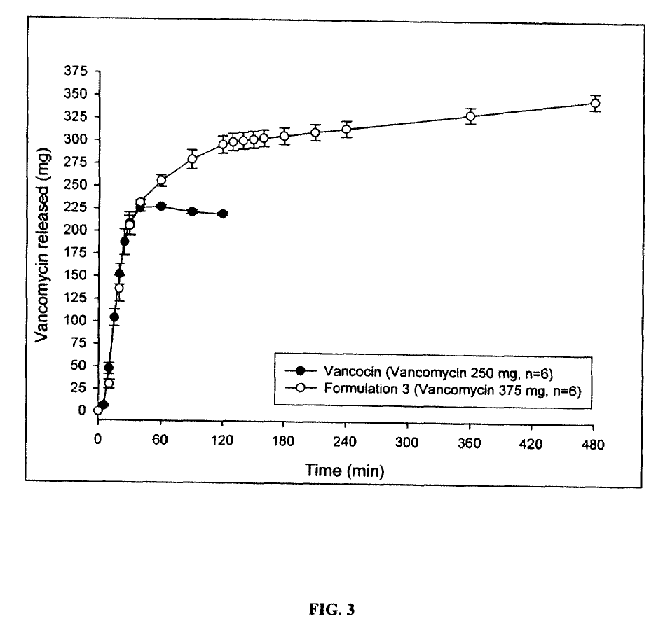

The following compositions have been prepared using the methods described herein. Dissolution results of example compositions are presented in FIG. 3.

TABLE 3
Formulation 3

| Dosage Form Component | Ingredients (in mg) | |
|---|---|---|
| Sustained Release Component | Vancomycin HC1 | 204 |
| | Gelucire | 296 |
| | Sodium Alginate | 90 |
| | TOTAL | 590 |
| Immediate Release Component | Vancomycin HC1 | 204 |
| | PEG400 | 96 |
| | Gelucire | 90 |
| | TOTAL | 390 |

Manufacturing Process of Formulation 3

Step A (Preparation of the Sustained Release Element): 1. Melt 11.84 grams of Gelucire® in a beaker in a hot plate stirrer at about 70° C. 2. Homogeneously disperse 8.16 grams of vancomycin HCI in the same beaker while maintaining at the same temperature. 3. Add 3.6 grams of sodium alginate into the same beaker and stir until homogeneous. 4. While warm, add 590 mg of the mixture into the bottom of a hard gelatin capsule. 5. Let the temperature of the fill drop to about room temperature.

Step B (Preparation of the Immediate Release Element): 6. Add and melt 3.84 grams of PEG400 and 3.6 grams of Gelucire(t in a beaker at a temperature of about 70° C. Mix the solution until homogeneous. 7. Add and disperse 8.16 grams of vancomycin HCI into the same beaker until homogeneous while maintaining the temperature at about 70° C. 8. Weigh 390 mg of the homogeneous suspension, and fill into the same hard gelatin capsule. 9. Allow the formulation to cool down to about room temperature. 10. Cap the capsules.

Formulation 3a

| Dosage Form Component | Ingredients (in mg) | |
|---|---|---|
| Sustained Release Component | Vancomycin HC1 | 153 |
| | PEG1500 | 82 |
| | Gelucire | 77 |
| Barrier | Paraffin | 33 |
| Immediate Release Component | Vancomycin HC1 | 153 |
| | Gelucire | 214 |
| | Sodium Alginate | 60 |

Manufacturing Process of Formulation 3a

Step A (Preparation of the Sustained Release Element): 1. Melt 36.38 grams of Gelucire in a beaker in a hot plate stirrer at about 70° C. 2. Homogeneously disperse 26.01 of Vancomycin HCl in the same beaker while maintaining at the same temperature. 3. Add 10.2 grams of sodium alginate into the same beaker and stir until homogeneous. 4. While warm, add 427 mg of the mixture into the bottom of a hard gelatin capsule. 5. Let the temperature of the fill drop to about room temperature.

Step B (Preparation of the Barrier Element): 6. Melt 30 grams of paraffin in a beaker in a hot plate stirrer at about 70° C. 7. While warm, add 33 mg of paraffin liquid, and fill into the same hard gelatin capsule. 8. Allow the formulation to cool down to about room temperature.

Step C (Preparation of the Immediate Release Element): 9. Add and melt 13.94 grams of PEG 3500 and 13.09 grams of Gelucire in a beaker at a temperature of about 70° C. Mix the solution until homogeneous. 10. Add and disperse 26.01 grams of vancomycin HCl into the same beaker until homogeneous while maintaining the same temperature. 11. Weight 312 mg of the homogeneous suspension, and fill into the same hard gelatin capsule. 12. Allow the formulation to cool down to about room temperature. 13. Cap the capsules.

### Example 4

Quantitation of vancomycin in solution can be determined by either a HPLC method or an UV spectrometry method. Example conditions for each method are provided below.

a. HPLC Methods

The HPLC conditions are described as follows: Column: Kromasil 5 μ 100A C18, 4.6×250 mm; Mobile phase: ACN-50 mM NaH₂PO₄ H₂O=10:90 (v/v). Adjusted to pH 7.0 with 10 N NaOH; Flow rate: 1.0 mL/min; Wavelength: 280 nm; Volume of injection: 20 μL; Run time: 16 min

b. UV Spectrometry Methods

Wavelength: 280 nm; Pathlength: 1 cm

### Example 5

The performance of Vancocin® and an example sustained release formulation of the present invention have been evaluated using the methods described herein. The dissolution test of the formulation can be conducted by using the US Pharmacopoeia XXIII, Method I, in a basket apparatus at 100 rpm and temperatures at 37° C.

For Vancocin®, the dissolution study is conducted in a dissolution medium of 900 mL of a simulated gastric fluid of 0.IN HCl (pH 1.0) for one hour. Samples, 3 mL each, were taken at 5, 10, 15, 20, 25, 30, 40 and 60 minutes. An equal volume of fresh medium is replaced into the dissolution vessel after each sample withdrawal. After each sampling, the samples are assayed by a HPLC method.

For the sustained release formulations, the dissolution studies are conducted in a dissolution medium of 900 mL of a simulated gastric fluid of 0.1N HCl (pH 1.0) for 2 hours initially. Samples are detected by an on-line UV spectrometry at 10, 20, 30, 40, 60, 90 and 120 minutes. After 2 hours, the dissolution medium is decanted from the dissolution vessel and is replaced by a dissolution medium of 900 mL of simulated intestinal fluids (pH 6.8) for another 6 hours. Samples are detected by an on-line UV spectrometry at 10, 20, 30, 40, 60, 90, 120, 240 and 360 minutes. Wavelength of detection is set at 280 nm.

We claim:

1. A pharmaceutical composition comprising an antibiotic, wherein the composition is a sustained release dosage form comprising at least one immediate release component and at least one sustained release component.

2. The composition of claim 1, wherein the sustained release dosage form is a capsule.

3. The composition of claim 1, wherein the immediate release component comprises at least one hot-melt excipient that is dissolved in an aqueous media and in liquid form at temperatures above 35° C.

4. The composition of claim 1, wherein the sustained release component comprises at least one hot-melt excipient that is dissolved in an aqueous media and in liquid form at temperatures above 35° C.

5. A pharmaceutical composition comprising vancomycin, wherein the composition is a sustained release dosage form comprising at least one immediate release component and at least one sustained release component.

6. The composition of claim 5, wherein the vancomycin is vancomycin HCI.

7. The composition of claim 5, wherein the composition comprises about 150 mg to 520 mg of vancomycin HCl.

8. The composition of claim 5, wherein the sustained release dosage form is a capsule.

9. The composition of claim 5, wherein the immediate release component comprises at least one hot-melt excipient that is dissolved in an aqueous media and in liquid form at temperatures above 35° C.

10. The composition of claim 9, wherein the hot-melt excipient is selected from the group consisting of polyethylene glycol esters and polyethylene glycols.

11. The composition of claim 5, wherein the sustained release component comprises at least one hot-melt excipient that is dissolved in an aqueous media and in liquid form at temperatures above 35° C.

12. The composition of claim 11, wherein the hot-melt excipient is selected from the group consisting of polyethylene glycol esters and polyethylene glycols.

13. The composition of claim 5, wherein the sustained release component further comprises a pH sensitive ingredient which is not soluble in acidic environment but is soluble in an alkaline environment.

14. The composition of claim 13, wherein the pH sensitive ingredient is selected from the group consisting of sodium alginate and sodium carboxymethyl cellulose.

15. The composition of claim 5, wherein about 50% of the vancomycin is released in one hour.

16. The composition of claim 5, wherein about 50% of the vancomycin is released between about one hour and about two hours.

17. The composition of claim 5, wherein substantially all of the vancomycin is released over a period of twelve hours.

18. A method of treating enter-colitis, the method comprising administering an effective amount of the pharmaceutical composition according to claim 5 to a subject in need thereof.

19. The method of claim 18, wherein the pharmaceutical composition is taken less than three times a day.

20. The method of claim 18, wherein the method provides a minimum inhibitory concentration of vancomycin in a subject.

21. The method of claim 18, wherein the method delivers the vancomycin locally within the gastrointestinal tract.

22. A method for preparing a capsule formulation of an antibiotic, the method comprising filling the capsule with at least one immediate release component and at least one sustained release component.

23. The method of claim 22, wherein the immediate release component and sustained release components are added in separate filling steps.

24. The method of claim 22, wherein the capsule formulation comprises at least one hot-melt excipient that is dissolved in an aqueous media and in liquid form at temperatures above 35° C.

25. The method of claim 22, wherein the antibiotic is vancomycin.
